(19) United States
(12) Patent Application Publication
(10) Pub. No.: US 2007/0050001 A1
(43) Pub. Date: Mar. 1, 2007

(54) Adjustable open loop control devices and methods

(75) Inventors: Edward Luttich, Dublin, CA (US); Abdul M. Tayeb, San Leandro, CA (US); Oren A. Mosher, Castro Valley, CA (US)

Correspondence Address: OPPENHEIMER WOLFF & DONNELLY LLP, 45 SOUTH SEVENTH STREET, SUITE 3300, MINNEAPOLIS, MN, 55402, US

(73) Assignee: SOLARANT MEDICAL, INC., Livermore, CA (US)

(21) Appl. No.: 11/213,567

(22) Filed: Aug. 26, 2005

(51) Int. Cl. A61F 2/00

(52) U.S. Cl. 607/102

(57) ABSTRACT

Noninvasive methods for therapeutically heating a collagenous structural support tissue of a pelvic support system to a desired temperature range are provided. One method comprises delivering energy to the structural support tissue to heat the tissue to the desired temperature range by ramping up a power level for a first period of time. A first constant high power level is then maintained for a second period of time. The power level is then ramped down for a third period of time. A second constant lower power level is then maintained for a fourth period of time. This power application treatment yields a favorable heat treatment maximizing predictability and efficacy while maintaining sufficient levels of safety. Further, such open loop power algorithms advantageously provide control without the need for subsidiary tissue temperature feedback measurements from sensors or tissue penetrating needles.

### BACKGROUND OF THE INVENTION

The present invention generally relates to medical systems, methods, and software. More specifically, the present invention provides adjustable open loop control systems, methods, and software for selectively heating tissues, particularly for the noninvasive treatment of urinary incontinence.

Urinary incontinence arises in both men and women with varying degrees of severity, and from different causes. In men, the condition frequently occurs as a result of prostatectomies which result in mechanical damage to the urinary sphincter. In women, the condition typically arises after pregnancy when musculoskeletal damage has occurred as a result of inelastic stretching of the structures supporting the genitourinary tract. Specifically, pregnancy can result in inelastic stretching of the pelvic floor, the external sphincter, and the tissue structures which support the bladder, urethra, and bladder neck region. In each of these cases, urinary leakage typically occurs when a patient's abdominal pressure increases as a result of stress, e.g., coughing, sneezing, laughing, exercise, or the like.

Treatment of urinary incontinence can take a variety of forms. Most simply, the patient can wear absorptive devices or clothing, which is often sufficient for minor leakage events. Alternatively or additionally, patients may undertake exercises intended to strengthen the muscles in the pelvic region, or may attempt a behavior modification intended to reduce the incidence of urinary leakage.

In cases where such non-interventional approaches are inadequate or unacceptable, the patient may undergo surgery to correct the problem. A wide variety of procedures have been developed to correct urinary incontinence in women. Several of these procedures are specifically intended to support the bladder neck region. For example, sutures, straps or other artificial structures are often looped around the bladder neck and affixed to the pelvis, the endopelvic fascia, the ligaments which support the bladder, or the like. Other procedures involve surgical injections of bulking agents, inflatable balloons, or other elements to mechanically support the bladder neck.

In work done related to the present invention, it has been proposed to treat urinary incontinence by selectively remodeling a portion of the pelvic support tissue, often so as to reposition the bladder and/or urogenital tract. U.S. Pat. No. 6,091,995 generally describes laparoscopic and other minimally invasive devices, methods, and systems for shrinking tissues, particularly for treatment of incontinence. U.S. Pat. Nos. 6,216,704; 6,558,381; and 6,546,934, describe noninvasive devices, methods, and systems for shrinking of tissues, often by cooling a surface of an intermediate tissue and directing energy through the cooled intermediate tissue to the target tissue so as to effect shrinkage. U.S. Pat. Nos. 6,156,060; 6,572,639; and 6,776,779, are directed to static devices and methods to shrink tissues for incontinence. Finally, U.S. Pat. No. 6,292,700 describes an endopelvic fascia treatment for incontinence in which a strength of a collagenous tissue increases, optionally without collagenous tissue contraction. U.S. patent application Ser. No. 10/759,732, filed Jan. 15, 2004, describes non-surgical incontinence treatment systems and methods. Each of these patents is assigned to the assignee of the present application, and their full disclosures are incorporated herein by reference.

While these recent proposals for treatment of incontinence represent significant advancements in the art, treatment of incontinence and other conditions related to insufficient collagenous tissue support could benefit from still further advances. For example, temperature sensing mechanisms such as tissue penetrating needles for feedback control may lead to burns on non-target healthy tissues. Temperature sensing needles may also not effect complete heating of target tissue due to a “tenting” effect caused by trapped air and fluid pockets which act to reduce thermal conductivity. For these reasons, it would be desirable to provide improved adjustable open loop control systems, methods, and software for selectively heating support tissues of the body. It would further be desirable if these improved systems and methods provide for truly noninvasive therapy for these support tissues, especially for the treatment of urinary incontinence in men and women. It would be still further desirable if these improved systems and methods provide a good ratio of both tissue treatment efficacy and safety while being less complex and costly to manufacture.

### BRIEF SUMMARY OF THE INVENTION

The present invention provides improved adjustable open loop power control systems, methods, and software for selectively heating fascia, tendons, and other support tissues of the body to a desired temperature range. In particular, the systems, methods, and software of the present invention control the delivery of a therapeutic energy that can heat and strengthen a collagenous structural support tissue within a pelvic support system. Advantageously, methods and systems of the present invention eliminate reliance on temperature sensors or tissue penetrating needles for control feedback, and as such provide a truly noninvasive therapy for support tissues, especially for the treatment of urinary incontinence in men and women. Such noninvasive systems are further simpler, more reliable and less costly to manufacture. It will further be appreciated that the present invention is not limited to incontinence therapy, but may also be applied to a variety of conditions such as bladder neck descent, hernias, cosmetic surgery, and the like. As discussed in more detail below, the present invention provides methods, systems, and computer implemented open loop power algorithms that yield enhanced efficacy through improved tissue treatment volumes while maintaining sufficient safety zones and minimizing complications, such as needle burns.

In one aspect of the present invention, a method for therapeutically heating a collagenous structural support tissue of a pelvic support system to a desired temperature range is provided. The method comprises delivering energy to the structural support tissue to heat the tissue to the desired temperature range by ramping up a power level for a first period of time. A first constant high power level is then maintained for a second period of time. The power level is then ramped down for a third period of time. A second constant lower power level is then maintained for a fourth period of time. This power application treatment yields favorable heat treatment temperatures maximizing predictability and efficacy while maintaining sufficient levels of safety.

A ramping up of the power level for the first period of time may comprise ramping up an initial starting power level of no greater than 22 watts, preferably no greater than 16 watts at a slope of no greater than 0.5 watts per second, preferably no greater than 0.25 watts per second. The first period of time may be in a range from 50 seconds to 220 seconds. The first constant high power dwell may be in a range from 34 watts to 40 watts, preferably no greater than 38 watts and the second period of time may be in a range from 60 seconds to 200 seconds. Ramping down of the power level for the third period of time may comprise ramping down the power level to a range from 29 watts to 33 watts at a slope in a range from 0.5 watts per second to 20 watts per second. The third transition period of time may be in a range from 1 second to 10 seconds, typically less than 3 seconds. The second constant low power dwell may be in a range from 29 watts to 33 watts, preferably 30 watts and the fourth period of time may be in a range from 15 seconds to 120 seconds.

Such open loop power methods result in heating the structural support tissue to the desired temperature range between 54° C. and 76° C. with improved predictability. The energy delivery patterns produce a mean minimum safety zone thickness in an intermediate tissue of at least 0.3 mm, preferably at least to 0.5 mm. The energy delivery patterns further produce a mean predominant safety zone thickness in an intermediate tissue of at least 0.5 mm, preferably at least 1.0 mm. The energy delivery patterns also provide enhanced efficacy by producing a tissue treatment volume in a range from 1 cubic centimeters to 5 cubic centimeters. An effective thermal capacity of the tissue treatment volume, denoted by capital letter Q herein, may be in a range from 40 joules/° C. to 87 joules/° C. A coefficient of thermal conductivity between a measured point in the tissue treatment volume and a non-treated tissue, denoted by the capital letter D herein, is in a range from 0.39 watts/° C. to 1.19 watts/° C. A coefficient of thermal conductivity between a measured point in the tissue treatment volume and an applicator body, denoted by the capital letter K herein, is in a range from 0.2 watts/° C. to 0.35 watts/° C.

The energy preferably comprises radio frequency energy, however other forms of heating energy may be adapted to the principles of the present invention, such as electro-resistive, sound, infra-red, radiation, and like energies which may be projected into a subsurface body of the tissue. In some embodiments, the structural support tissue may be cooled by conductive surface cooling. In such instances, a cooled electrode applicator may deliver at much higher power levels than a non-cooled electrode applicator since the tissue heating effect is the net of heating power less the heat removed by cooling. The energy may be delivered so as to effect shrinkage of the structural support tissue and/or to cause bulking and buttressing of the structural support tissue during healing. Tissue strengthening via shrinkage or tissue bulking/buttressing inhibit urinary incontinence or bladder neck descent, wherein the structural support tissue may comprise a collegenated tissue in an endopelvic fascia and the intermediate tissue may comprise vaginal mucosa. The structural support tissue may be accessed transvaginally or laparoscopically.

In another aspect of the present invention, a system for therapeutically heating a collagenous structural support tissue of a pelvic support system to a desired temperature range is provided. The system comprises an applicator body and a processor coupleable to the applicator body. The processor may be programmed to deliver energy to the structural support tissue with the applicator body by ramping up a power level for a first period of time, maintaining a high power dwell for a second period of time, ramping down the power level for a third period of time, and maintaining a low power dwell for a fourth period of time. The system may further comprise a power supply coupleable to the processor as well as a cooling source coupleable to the processor.

In yet another aspect of the present invention, a computer-readable storage medium having a computer-readable program embodied therein for directing operation of a computer system is provided. The computer system including a communications system, a processor, and a memory device. The computer-readable program includes instructions for therapeutically heating a collagenous structural support tissue of a pelvic support system to a desired temperature range in accordance with the any of the method steps described herein.

In still another aspect of the present invention, a method and device for heating living human tissue to a prescribed temperature range is provided. This is accomplished by application of heating energy in a particular pattern (e.g., power level versus time) such that the inherent ability of the specific tissue to absorb and dissipate heat interacts with the specific applied power pattern to yield the prescribed temperature range. As such, the need to invasively measure the tissue temperature and employ feedback control is thereby circumvented.

A further understanding of the nature and advantages of the present invention will become apparent by reference to the remaining portions of the specification and drawings.

### BRIEF DESCRIPTION OF THE DRAWINGS

The following drawings should be read with reference to the detailed description. Like numbers in different drawings refer to like elements. The drawings, which are not necessarily to scale, illustratively depict embodiments of the present invention and are not intended to limit the scope of the invention.

FIG. 1 is a simplified system that includes a control unit and a noninvasive applicator which incorporate the principles of the present invention.

FIG. 2 illustrates a surface of the noninvasive applicator with three active electrodes and insulators between the electrodes.

FIG. 3A is a flow diagram of a control unit incorporating the principles of the present invention.

FIG. 3B is a flow diagram illustrating one embodiment of a method through which the principles of the present invention are employed in the energy delivery process.

FIG. 4 is a graph which illustrates an exemplary open loop power algorithm in accordance with the principles of the present invention and the resulting tissue temperature curves achieved from in vitro studies.

FIG. 5 illustrates the noninvasive applicator heating tissue.

FIG. 6 illustrates the characteristic heat plume during heating of tissue.

FIG. 7 is a graph which illustrates another exemplary open loop power algorithm and the theoretically predicted tissue temperature response curve.

FIG. 8 is a graph which illustrates observed tissue temperature curves from in vitro studies versus theoretically predicted tissue temperature curves.

FIG. 9 illustrates a transvaginal noninvasive applicator heating collegenated tissue in an endopelvic fascia through vaginal mucosa intermediate tissue.

FIG. 10 is a graph which illustrates another exemplary open loop power algorithm and theoretically predicted tissue temperature response curves.

FIGS. 11A through 11C are graphs which illustrate safety zone thickness in tissue from in vitro treatment temperature studies.

FIG. 12 is a graph which illustrates tissue safety zone thickness and tissue treatment volumes from in vitro treatment temperature studies.

FIGS. 13A and 13B are graphs which illustrate further tissue treatment volumes from in vitro treatment temperature studies.

FIG. 14 is a graph which illustrates an equilibrium experiment to measure a thermal coefficient of conductivity K.

FIG. 15 is a graph which illustrates an experiment with a non-cooled applicator to measure an effective thermal capacity Q.

FIG. 16 is a graph which illustrates further observed tissue temperature curves from in vitro studies versus theoretically predicted tissue temperature curves.

FIGS. 17A through 17D illustrate alternative power profiles that may be utilized to achieve results similar to those of the present invention.

### DETAILED DESCRIPTION OF THE INVENTION

The present invention provides methods, systems, and software algorithms for controlling delivery of energy to a body's support tissue to enhance the structural support provided by the body's support tissues. The present invention may be directed to inducing controlled stiffening, contraction, or shrinkage of the structural support tissue of the body, typically being a collagenous tissue such as fascia, ligament, or the like.

For example, in one specific use, the present invention provides for treatment of urinary incontinence. The structural support tissue will be part of a pelvic support system that is responsible in some manner for control of urination, or for supporting such a tissue. The tissues of the pelvic support system generally maintain the position of the genitourinary tract, and particularly the position of urinary bladder, urethra, and the bladder neck coupling these structures. In general, endopelvic fascia may define a hammock-like structure which extends laterally between the left and right arcus tendineus fasciae pelvis (ATFP). These tendon structures may extend substantially between the anterior and posterior portions of the pelvis, so that the endopelvic fascia EF at least partially defines the pelvic floor.

The fascial tissue of the pelvic support system may comprise tissues referred to under different names by surgeons of different disciplines, and possibly even by different practitioners within a specialty. In fact, some surgeons may assign a collagenous support structure of the endopelvic fascia one name when viewed from a superior approach, and a different name when viewed from an inferior approach. Some of the endopelvic fascia may comprise two collagenous layers with a thin muscular layer therebetween, or may comprise a single collagenous layer. The hammock-like endopelvic fascia described herein may be damaged or missing, particularly after pregnancy, so that the support of the genitourinary tract is instead provided by a variety of fascial layers, muscular tissues, ligaments, and/or tendons within the pelvis. Hence, the treatment of the present invention may be directed at a variety of tissue structures defining the pelvic floor and/or diaphragm (including: anterior sacro-coccygeal ligament; arcus tendineus fasciae pelvis ATFP, the white line of the pelvis; fasciae of the obturator intemus muscle; the arcus tendineus levator ani or “picket fence” to the iliococcygeus portion of the levator ani muscle; bulbocavernosus muscle; ischiocavemosus muscle; urethrovaginal sphincter; m. compressor urethrae muscle; and m. sphincter urethrovaginal muscle which replaces deep perineal muscle); structures of the bladder and urethra (including: urethrovesical fascia; detrusor muscle; and the pubococcygeus muscle which relaxes to open the bladder neck, initiating micturation); structures of the vagina (including: vagino-uterine fascia, lamina propria—the dense connective tissue layer just under the epithelium; pubo-urethral or puboprostatic ligaments; pubo-vesicle ligament and posterior pubo-urethral or puboprostatic ligament; pubovesicle muscle, a smooth muscle that is integrated with the pubovesicle ligament; and pubocervical fascia which attaches to the ATFP); structures of the uterus (including: round ligament; sacrouterine ligament; and broad ligament); and structures of the bowel (including: rectal fascia and mackenrodt's ligament).

When the endopelvic fascia has excessive length or stretches excessively under a load, the fluid pressure within the bladder advances into the bladder neck and down the urethra more readily. Leakage may result in part because the endopelvic fascia allows the bladder, bladder neck, and/or urethra to drop below its desired position, at which fluid pressure within the bladder may actually help to seal the bladder neck. Stretching of the endopelvic fascia may also alter the timing of pressure pulse transmission to the urethra.

When a continent woman coughs, the pressure in the urethra will often increase more than one-tenth of a second prior to the increase in bladder pressure. In women with stress incontinence, the bladder pressure may rise first. For a continent woman having endopelvic fascia which stretches much less under the influence of a pressure pulse, the time delay between initiation of the pressure pulse and transferring sufficient force to urethra to effect closure may therefore be significantly less. By treating the endopelvic fascia to decrease its length and/or increase its stiffness, the descent time of the pelvic viscera during a cough will be shorter than an untreated, excessively long and/or excessively elastic tissue.

The support tissue may be treated non-surgically or it may be accessed for direct treatment in a variety of ways. When using a multi-electrode applicator, for example, the surface of the endopelvic fascia (or other tissue) may be accessed transvaginally by forming and displacing a flap from the vaginal wall with the assistance of a weighted speculum. Alternatively, the endopelvic fascia may be accessed laparoscopically. When using the noninvasive cooled electrode applicator, the tissue may be accessed directly by placing the applicator on the anterior vaginal wall.

Tissue contraction or stiffening results from controlled heating of the tissue by affecting the collagen molecules of the tissue. Contraction occurs as a result of heat-induced uncoiling and repositioning of the collagen β-pleated structure. By maintaining the times and temperatures set forth below, significant tissue contraction may be achieved. Tissue strengthening by controlled contraction or shrinkage is described in more detail in U.S. Pat. No. 6,836,688, assigned to the assignee of the present application and incorporated herein by reference. Stiffening results from the loss of elasticity of the tissue due to the formation of scar tissue and/or attachment of adjacent support tissues to each other as a result of controlled heating of the tissue. Tissue strengthening by bulking and buttressing of the structural support tissue due to the healing process and/or formation of scar tissue is described in more detail in U.S. Pat. No. 6,292,700, assigned to the assignee of the present application and incorporated herein by reference.

While the remaining description is generally directed to a system for treatment of urinary stress incontinence of a female patient, it will be appreciated that the present invention will find many other applications for selectively directing therapeutic heating energy into the tissues of a patient body. For example, treatment of other conditions may be effected by selective ablation, shrinking or stiffening of a wide variety of other tissues, including (but not limited to) the diaphragm, esophagus, the nasal concha, the abdominal wall, the breast supporting ligaments, the fascia and ligaments of the joints, the collagenous tissues of the skin, tumors, and the like.

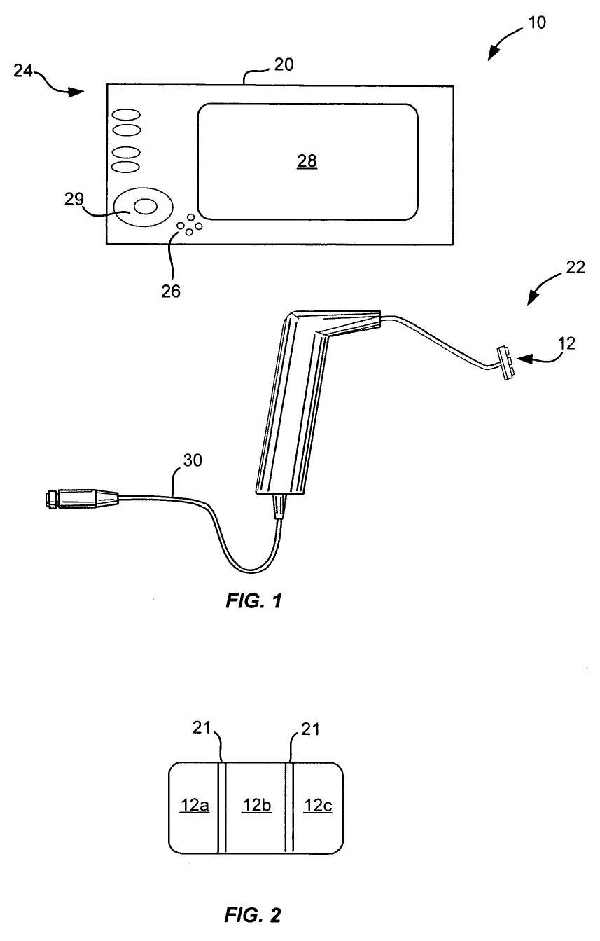

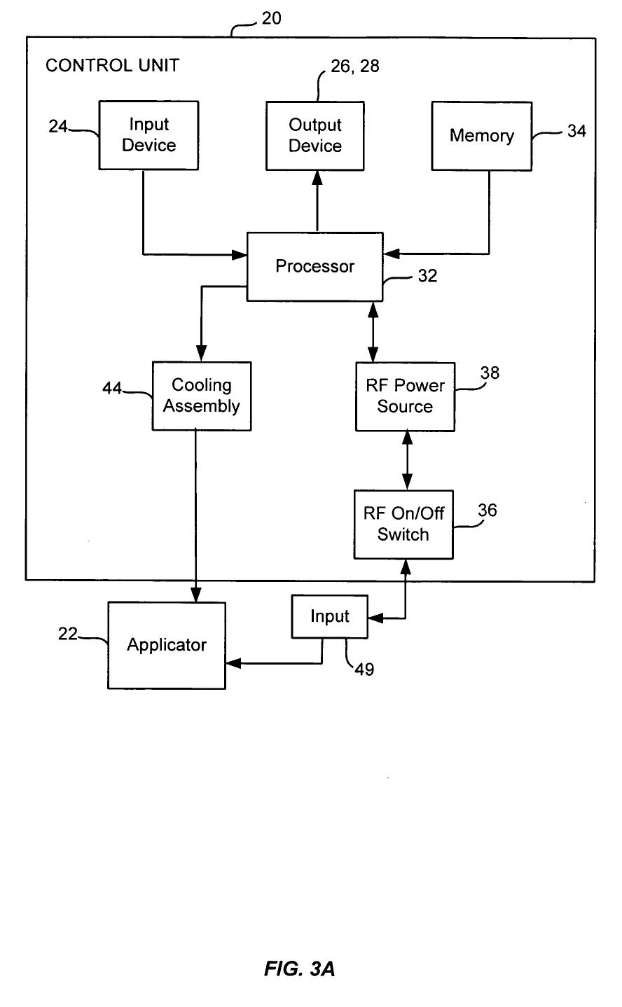

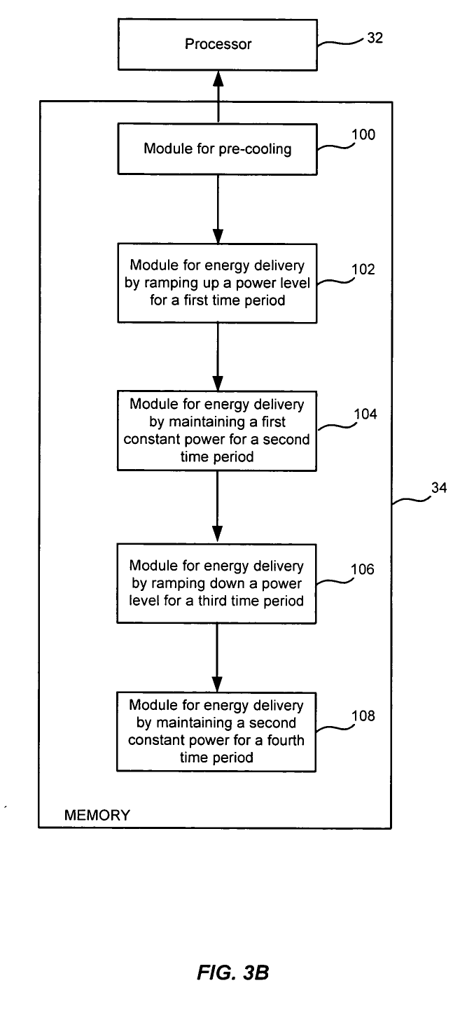

FIG. 1 illustrates a simplified system 10 that incorporates the principles of the present invention. System 10 generally includes a control unit 20 that controls a delivery of energy to electrodes 12 on a noninvasive applicator body 22. Control unit 20 includes input device(s) 24, output device(s) 26, and a display device 28. Applicator 22 is attached to an output 29 of control unit 20 via a coupler 30 that may contain one or more couplings. Applicator 22 may include one or more input devices 49 (FIG. 3A), such as a trigger or foot pedal, for activating the delivery of energy. A distal end of applicator 22 may be shaped to laparoscopically or transvaginally access the support structure tissue.

FIG. 2 illustrates a substantially flat surface of the noninvasive applicator 22 that is encompassed by the present invention. One or more input devices, such as a footswitch (not shown) or trigger 49, may be coupled to applicator 22 and in communication with switch 36 (FIG. 3A) to control the delivery of energy through applicator 22. Applicator 22 may have a three electrode configuration 12a, 12b, 12c, that are separated by insulators 21 to deliver the energy to the tissue. Applicator 22 may take on a variety of different sizes and shapes. In the noninvasive cooled electrode embodiment, applicator 22 preferably has a diameter of between about 2 cm and about 4 cm, a treatment surface that has a width between 2 cm and 3 cm and a length between about 3 cm and about 5 cm long, and a shaft length of between about 6 cm and about 12 cm. For example, the treatment surface in FIG. 2 is 25 mm wide by 39 mm long and has 1 mm long insulators 21 between the electrodes 12a, 12b, 12c.

While three active electrode segments are illustrated in FIG. 2, it should be appreciated that any number of electrode segments may be used. For example, in other embodiments there may be a single pair of electrodes. Some applicators and systems that may be used to deliver the energy are described in U.S. patent application Ser. No. 09/229,508, filed Jan. 12, 1999, U.S. Patent Application Ser. No. 60/440,711, filed Jan. 16, 2003, U.S. patent application Ser. No. 10/102,596, filed Mar. 19, 2002, U.S. patent application Ser. No. 10/759,732, filed Jan. 15, 2004, and U.S. Pat. No. 6,216,704, all assigned the assignee of the present application and the full disclosures of which are incorporated herein by reference.

FIG. 3A is a flow diagram of control unit 20. In preferred embodiments, control unit may be of a size and shape that allows the control unit 20 to be mounted on a standard hospital IV pole. Control unit 20 includes a processor 32 that controls the functionality of control unit 20. Processor 32 has associated therewith a memory 34 adapted to store software code instructions to operate the assemblies in control unit 20 so as to carry out the methods of the present invention. Input devices 24, such as one or more buttons, are coupled to processor 32 to allow a user to input data and instructions into control unit 20. One or more output devices 26, such as a speaker, are coupled to processor 32 to allow audible tones to be output to the user during the procedure to provide treatment information to the user. A display 28 cooperates with processor 32 to provide visual status and error messages pertaining to each step of the process carried out by the present invention.

Control unit 20 includes a switch 36 that serves to activate and deactivate transmission of energy from a power source 38, such as a bipolar radiofrequency (RF) power source, to electrodes 12 on applicator 22. Switch 36 may be activated with activation and deactivation of the input device 49 on applicator 22, or the like. The power source 38 is coupled to the processor 32. In the three electrode configuration of FIG. 2, the control unit 20 applies bipolar radio frequency energy alternately between the center electrode segment 12b and either the distal electrode segment 12a or the proximal electrode segment 12c.

A cooling assembly 44 may optionally be coupled to processor 32 and applicator 22 and will be configured to pre-cool the tissue contacted by applicator 22 and/or cool the tissue during the delivery of the energy. A more complete description of some examples of cooling assembly 44 are described in commonly owned U.S. Pat. Nos. 6,091,995 and 6,480,746, the complete disclosures of which are incorporated herein by reference. As can be appreciated, cooling assembly 44 is optional and not all applicators of the present invention include cooling assembly 44.

Processor 32 may identify and display appropriate error messages pertaining to a variety of conditions, such as errors encountered during the diagnostic system tests, and the like. Some embodiments of processor 32 allow the user to set date and time, audio tone level, language selection for display on display device 28, power levels, desired treatment times, desired temperature goals, desired safety zone thickness, desired treatment volume, and the like. In some embodiments, such parameters may be preset. Processor 32 generates audio tones to prompt the user for actions and to indicate error and out of range conditions. A continuous or intermittent audio tone may be emitted by a speaker 26 associated with processor 32 at a steady rate when energy is applied. Processor 32 may generate a welcome screen showing a logo or other graphics desired by user of system 10. Processor 32 may display recoverable error condition messages and prompts the user to correct the cause. Unrecoverable error messages may be displayed on display device 28 and give appropriate error information.

Control unit 20 may be configured to complete a self-test each time the power source 38 is turned on. Control unit 20 allows processor 32 to complete its internal tests and display error messages accordingly. A fault in the power source output test can be diagnosed and displayed as an error condition. Processor 32 may be programmed to provide a clock signal for hardware detection of software operation. Processor 32 performs tests of internal subsystems, including but not limited to the analog and digital electronics. Control unit 20 provides a special test, diagnostics and service mode, which will allow the manufacturer or servicer of system 10 to bypass the normal diagnostic self-tests, be able to manually execute all functions and perform calibration and setup. This mode is generally not be accessible to the user.

FIG. 3B is a flow diagram illustrating code modules that may be stored in the memory 34 and processed by processor 32 to carry out one embodiment of a method through which the principles of the present invention are employed in tissue strengthening via contraction and/or stiffening of a support structure tissue of a pelvic support system of a patient for treatment of incontinence using the cooled electrode applicator 22. Initially, the user may access the target support tissue and position the applicator 22 against tissue either transvaginally or laparscopically. At step 100, the structural support tissue comprising a collegenated tissue in an endopelvic fascia and the intermediate tissue comprising a vaginal mucosa may be pre-cooled. It will be appreciated that the pre-cooling step 100 is optional and in some instances unnecessary.

At step 102, energy is delivered to the structural support tissue comprising collegenated tissue in an endopelvic fascia to heat the tissue to a desired temperature range by starting at a low power application level and slowly ramping up for a first period of time to a peak applied power (first constant power level). This allows the resulting “heat plume” beneath the tissue surface to develop into a characteristic form, rather than having the heat concentrate in a very small volume because it can not dissipate as fast as the energy is being applied. This allows the device to heat the maximum volume of tissue for any applied peak temperature as well as yields a more consistent temperature response curve. A ramping up of the power level for the first period of time may comprise ramping up an initial power level that is no greater than 22 watts, preferably no greater than 16 watts at a slope that is no greater than 0.5 watts per second, preferably no greater than to 0.25 watts per second. The first period of time may be in a range from 50 seconds to 220 seconds. Typically, the inherent conductive cooling rate applied by the device is kept up with the rate of energy application so that the surface safety zone is maintained at a maximum for any given peak temperature. This is because the rate of conductive cooling rises with temperature. As such, it is acceptable to go to higher power levels after temperature is built up.

At step 104, the peak applied power or first constant high power level is then maintained for a second period of time. The peak power level is low enough so that the heating rate does not outrun the inherent conductive cooling rate of the device, in order to maintain the safety zone at the tissue surface. The peak power level is high enough and sustained for long enough so that, together with the ramp period, it achieves the desired temperature range in an acceptable period of time. This peak power level will be above the equilibrium power rate required to sustain the target temperature range. The desired temperature range is determined by the need to heat as much volume as possible to tissue necrosis temperature (50° C.) and to achieve as high a volume of collagen shrinkage as possible, which is a time-temperature dependent effect, while keeping peak temperature below the maximum safe value for all patients. The actual practical peak temperature range may be determined by the variation in tissue thermal characteristics across the patient population, as these characteristics interact with a fixed power algorithm to achieve a range of outcomes. Typically, the first constant high power dwell may be in a range from 34 watts to 40 watts, preferably 35 watts and the second period of time may be in a range from 60 seconds to 200 seconds. Typically at the high power dwell level, an equilibrium temperature is above the desired peak temperature so that the desired temperature may be reached rapidly.

At step 106, once the peak power period (first constant high power level) is completed, the power level is then ramped down from the peak applied power for a third period of time to a level closer to the equilibrium power level required to maintain the target temperature range (second constant lower power level). Ramping down of the power level for the third period of time may comprise ramping down the power level to a range from 29 watts to 33 watts at a slope in a range from 0.5 watts per second to 20 watts per second. The third transition period of time may be in a range from 1 second to 10 seconds, generally less than 3 seconds. At step 108, the second constant lower power level is then maintained for a fourth period of time which is practical to achieve maximum dwell time near the desired peak temperature. The second constant low power dwell as noted above may be in a range from 29 watts to 33 watts, preferably 30 watts and the fourth period of time may be in a range from 15 seconds to 120 seconds. Due to the variability of tissue response, the range chosen for the second constant lower power level may cause some patient treatments to slightly rise in temperature while others may slightly fall in temperature and while others may remain constant. Typically at the low power dwell level, the equilibrium temperature is closer to the desired peak temperature.

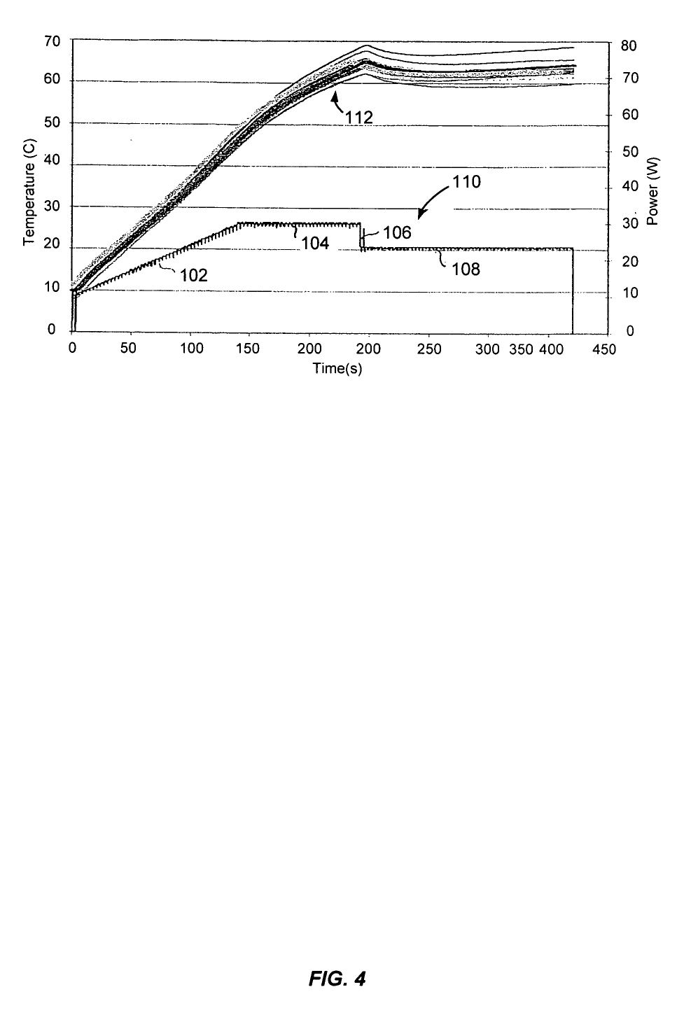

FIG. 4 illustrates that this open loop power application 110 of steps 102 through 108 yields favorable heat treatment temperatures maximizing predictability. Specifically, FIG. 4 illustrates twenty five resulting tissue temperature curves 112 achieved from in vitro studies on bovine liver tissues with the open loop power application 110. It will be appreciated that the heating algorithm 110 in FIG. 4 for liver samples has lower operating power parameters than those for human tissues (in vivo) due to heat loss effects in human tissues, which is described in more detail below. In particular, step 104 in FIG. 4 is defined by a high power dwell in a range from 20 watts to 40 watts, in this case 30 watts for 100 seconds, and step 108 is defined by a second low power dwell in a range from 10 watts to 33 watts, in this case 23.5 watts for 180 seconds. In FIG. 4, it can be observed for this specified set of treatments on liver samples that the maximum temperature is within a 10° C. range, roughly between 66° C. and 76° C. Such open loop power methods 110 generally result in heating the structural support tissue to the desired temperature range between 54° C. and 76° C. with improved predictability.

The open loop control system of the present invention accounts for a variety of multi-variable components so as to achieve the desired heat treatment. For example, lower power levels, gentle power ramps, and lower maximum tissue temperatures have been found to increase safety zone thickness of intermediate tissue, such as the vaginal mucosa. On the other hand, higher power levels, increased time at a given power level, or higher maximum tissue temperatures result in rising tissue treatment volumes of the endopelvic fascia. Energy delivery will also depend in part on which tissue structure is being treated, how much tissue is disposed between the target tissue and the electrode, and the ability of the tissue to accept and store power. The power levels used in the present invention will also vary depending on the electrode size, electrode spacing, and whether or not cooling is used. For example, a cooled electrode applicator may deliver at much higher power levels than a non-cooled electrode applicator since the tissue heating effect is the net of heating power less the heat removed by cooling.

Hence, power levels, desired treatment times, desired temperature goals, desired safety zone thickness, desired treatment volume, a patient's anatomy, and applicator configurations are factors to be considered so as to improve efficacy while maintaining sufficient levels of safety. Generally, the energy delivery patterns produce a mean minimum safety zone thickness in an intermediate tissue of at least 0.3 mm, preferably at least 0.5 mm. The energy delivery patterns further produce a mean predominant safety zone thickness in an intermediate tissue of at least 0.5 mm, preferably at least 1.0 mm. The energy delivery patterns also provide enhanced efficacy by producing a tissue treatment volume in endopelvic fascia in a range from 1 cubic centimeters to 5 cubic centimeters.

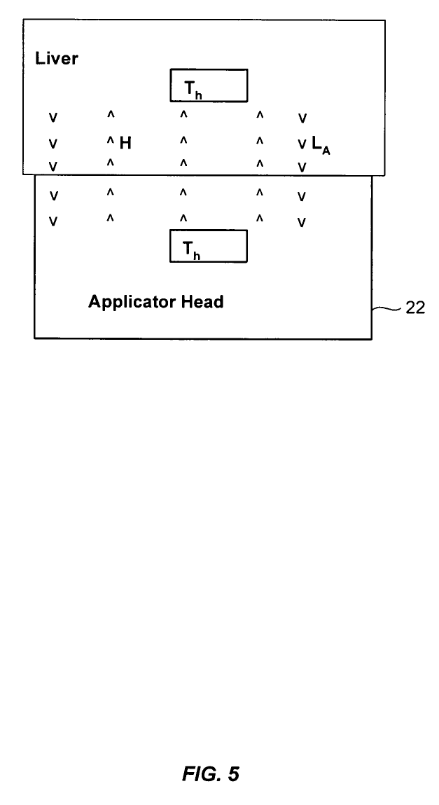

The theory of open loop RF power control is now described with reference to FIGS. 5 through 10. FIG. 5 illustrates in vitro studies of an applicator 22 heating sample liver tissue. Tₛ represents tissue temperature at a measured point (° C.), Tₛₑ represents tissue temperature at a measured point at equilibrium (° C.), Tₕ represents temperature of the applicator head 22 (° C.), H represents RF electromagnetic heating (watts or joules/sec), L_{A} represents cooling of tissue by the applicator head (watts or joules/sec), and K represents a coefficient of thermal conductivity between the measured temperature point Tₛ and the applicator head Tₕ (watts/° C.).

In these studies, heat losses due to the surroundings are assumed to be negligible. Therefore all heat flow is assumed to occur between the applicator head and the liver sample. Heat is applied through radio-frequency resistance heating. Cooling is by simple conduction due to the temperature difference between the tissue and the applicator head. For a given steady state RF power level and applicator head temperature, if the process is allowed to go on long enough, a characteristic equilibrium temperature will be reached. As the tissue is heated, cooling watts (L_{A}) increase with tissue temperature, while heating rate (H) remains constant, until cooling rate equals heating rate and the equilibrium temperature is reached. This may be represented by the following equations:
L_{A}=K(Tₛ−Tₕ)
at equilibrium H=L_{A}
H=L_{A}=K(Tₛₑ−Tₕ) or Tₛₑ=(H/K)+Tₕ (Equation #1)
H and Tₕ are set parameters, controlled by the applicator. K can be calculated from experimental data derived by running a heating cycle to equilibrium. In a typical test run, a steady state RF power application (H) of 20 watts with an applicator head temperature (Tₕ) of 0° C., yields an equilibrium tissue temperature (Tₛₑ) of 77° C. in about 45 minutes. Calculating K from Equation #1 yields 0.26 watts/° C.

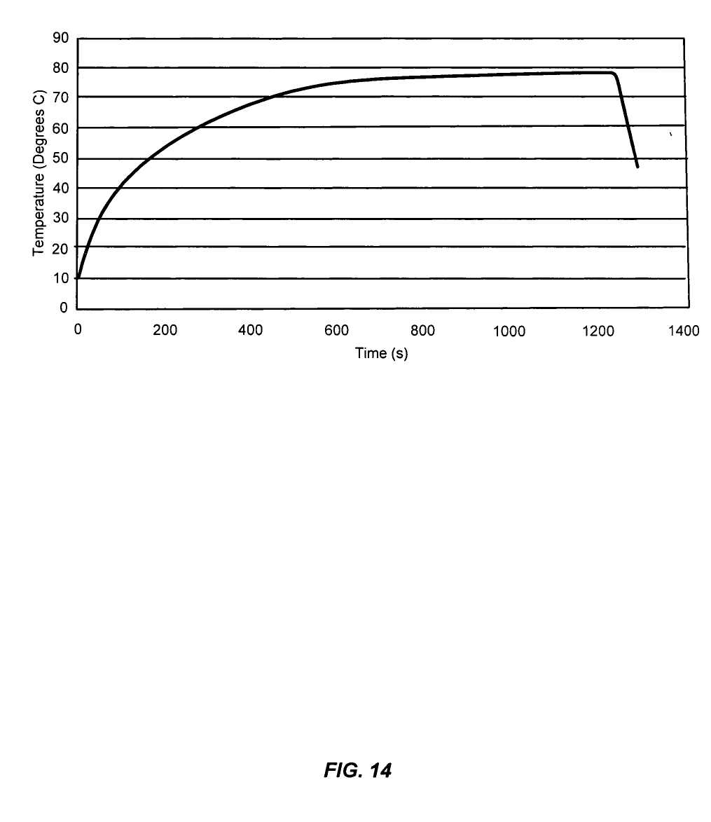

The value of K is determined by the nature of the applicator head and its materials as well as the power algorithm. Due to such factors, the value of K may be in a range from 0.2 watts/° C. to 0.35 watts/° C. in human tissues. It is a basic characteristic of the metal-to-tissue interface and is quite constant and similar between liver tissue samples and human tissue. Since the value of K can be arrived at through an equilibrium test (FIG. 14), its derivation is highly accurate and repeatable and as such may be treated almost like a fixed value. Unfortunately, equilibrium calculations are insufficient to describe power applications for incontinence treatment because 45 minute treatment procedures, per side, are lengthy and unpractical. The applicator therefore is operated at higher wattage levels to achieve the desired peak temperature ranges in much shorter time periods. Therefore non-equilibrium, time dependent effects to describe the behavior of the applicator need to be considered. After dealing with non-equilibrium factors, adaptation to other factors unique to the relevant human anatomy also need to be considered. Such factors include variation in the tissue's ability to store heat and losses of heat from the treatment site to the rest of the body.

Non-Equilibrium Factors

When a tissue sample is heated by the applicator 22, the equilibrium temperature will not be reached if one or both of the following conditions apply: (1) not enough time is allowed to elapse or (2) the equilibrium temperature exceeds the temperature at which the sample is altered or destroyed (in the treatment of incontinence, this corresponds to the occurrence of a “broad burn” which occurs around 80° C.). In the treatment of incontinence, the first condition prohibits reaching equilibrium as the lengthy treatment time is unpractical. The second condition should be avoided for safety reasons. Therefore the power algorithm selected after this characterization is complete, will be targeted to achieve a temperature range which tops out below 80° C.

In order to account for the time variation of temperature due to power input, we introduce another parameter Q which represents a thermal capacity of tissue volume being heated (joules/° C.). Q is a measure of the ability of the tissue to store heat. Conversely, Q together with the power input level, determines the rate of temperature rise. The basic geometry of the applicator head and the inherent characteristics of bipolar RF heating, cause the volume of tissue being heated to take on a very consistent characteristic geometry. This may be characterized by determining the 50° C. boundary. This is an elliptical cylindrical surface, where the tissue temperature is at 50° C. by the end of treatment. The 50° C. heat plume forms quickly during treatment and stabilizes, with further heating going into temperature rise within it. Outside the 50° C. heat plume, temperature drops off in a consistent steep gradient pattern. The 50° C. value is chosen because it is the approximate temperature at which tissue necrosis occurs.

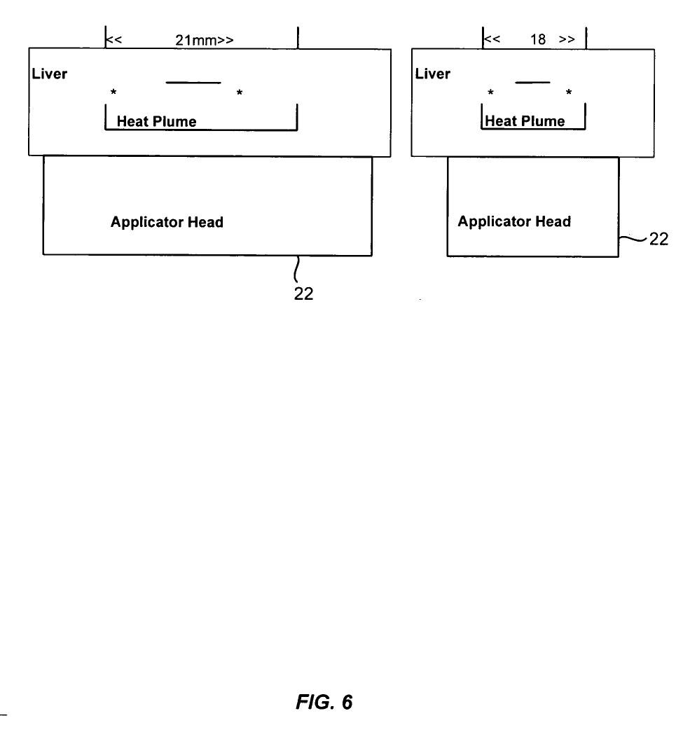

FIG. 6 illustrates the characteristic heat plume during heating of liver tissue with the applicator 22, the dimensions of the envelope being about 18 by 21 mm, varying only slightly with variations in the power algorithm. The depth varies slightly with the algorithm, but stays close to 10 mm. For algorithms which use ramped power to avoid abrupt heating, a meaningful effective value of Q for this characteristic heat plume can be developed. The value of Q depends physically on the volume being heated and the thermal capacity per unit volume, which is affected by moisture content, tissue density, and other factors. The calculation of Q may be inferred from relationships with other variables in observing test heating cycles.

Derivation of temperature versus time for constant RF power application will now be described. When operating the applicator on a liver tissue sample at a constant RF power level H, where heat losses are again assumed to be negligible, the instantaneous net heating which occurs is given by the following equations:
Hₙₑₜ=H−L_{A} or Hₙₑₜ=H−K(Tₛ−Tₕ)
The instantaneous rate of temperature rise is given by the following equations:
dTₛ/dt=Hₙₑₜ/Q or dTₛ/dt=(H−K(Tₛ−Tₕ))/Q
Solution of this differential equation is given in the form below:
Tₛ=Ce^{-(K/Q)t}+((H+KTₕ)/K)(1−e^{-(K/Q)t})
By inspection of boundary conditions, C=Tₒ (the starting temperature of tissue (° C.)) and solving the equations for Tₛ, the tissue temperature as a function of time may be derived for constant power by the following equation:
Tₛ=T₀e^{-(K/Q)t}+((H+KTₕ)/K)(1−e^{-(K/Q)t}) (Equation #2)
It will be appreciated that the for large values of time t, Equation #2 converges on Equation #1 as would be expected. If the Q value of a tissue sample is known, the curve of temperature versus time as the sample is heated by the applicator head at a constant RF power rate may be predicted by Equation #2.

To determine Q for the liver samples, a test heating at very low RF power may be performed, with the cooling system turned off. In this condition, the temperature of this sample rises in a straight sloped line, the slope of which is given by the following equations:
dTₛ/dt=H/Q or Q=H/slope
Knowing the H watts input, with a measurement of the slope of temperature versus time, Q may be in independently calculated. By the technique, it may be deduced that the effective Q for these liver samples is in a range from about 55 joules/° C. to 85 joules/° C. Determination of Q for human subjects varies over a wider range and will be accordingly deduced by other means as discussed below in the section on adapting the equations to human anatomy factors.

Derivation of temperature versus time for ramping RF power application will now be described. Since the actual open loop power algorithm used is segmented, with ramp-up and ramp-down periods as well as periods of level power, the tissue temperature as a function of time should be derived for a ramping power application. For RF power ramping at a slope P (watts/sec), at any given point in time, where Hₒ is the RF power at the beginning of the ramp, the instantaneous net heating which occurs is given by the following equation:
H=H₀+Pt
The instantaneous rate of temperature rise is given by the following equations:
dTₛ/dt=H/Q or dTₛ/dt=(H−K(Tₛ−Tₕ))/Q=((Ho+Pt)−KTs+KTh)/Q
Solution of this differential equation is given in the form below:
Tₛ=T0e^{(K/Q)t}+((H₀+KTₕ)/K)(1−e^{(K/Q)t})+(P/K)t−(PQ/K²)(1−e^{-(K/Q)t})
By inspection of boundary conditions, C=Tₒ (the starting temperature of tissue at the beginning of the RF power ramp (° C.)) and solving the equations for Tₛ, the tissue temperature as a function of time may be derived for a ramping RF power level by the following equation:
Tₛ=T₀e^{-(K/Q)t}+((H₀+KTₕ)/K)(1−e^{-(K/Q)t})+(P/K)t−(PQ/K²)(1−e^{-(K/Q)t}) (Equation #3)

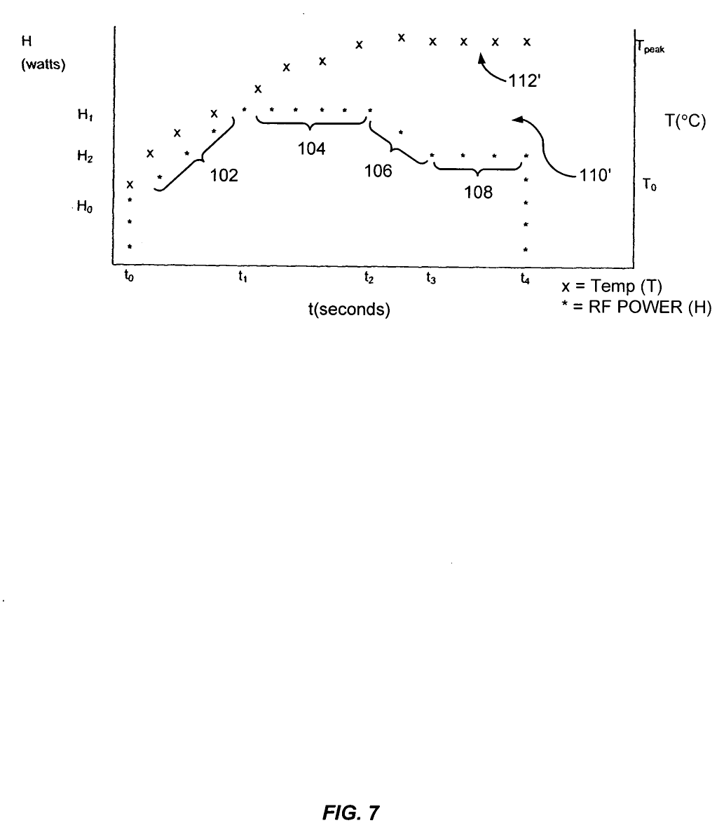

Temperature as a function of time for any RF power algorithm which is in the form of a series of ramped and level RF power periods may now be expressed for the liver tissue samples. The preferred open loop RF power algorithm 110′ is depicted in FIG. 7 by asterisk symbols (*). It comprises an RF power ramp-up period (step 102), followed by a dwell period at constant high power (step 104), followed by a ramp down period (step 106), followed by a dwell period of constant lower power (step 108).

Derivation of temperature versus time for a segmented power curve will now be described. In particular, the temperature versus time curve which results from the application of algorithm 110′ in FIG. 7 to a standard liver tissue sample may be expressed by the following equation from time t₀ to t₁:
T_{S}=T₀e^{-(K/Q)(t-t0)}+((H+KTₕ)/K)(1−e^{-(K/Q)(t-t0)})+(((H₁−H₀)/(t₁−t₀))/K)(t−t₀)−((H₁−H₀)/(t₁−t₀))Q/K²)(1−e^{-(K/Q)(t-t0)})
where H=H₀+(H₁−H₀)(t−t₀)/(t₁−t₀)
The temperature versus time curve which results from the application of algorithm 110′ in FIG. 7 to a standard liver tissue sample may be expressed by the following equation from time t₁ to t₂:
T_{S}=T₁e^{-(K/Q)(t-t1)}+((H₁+KTₕ)/K)(1−e^{-(K/Q)(t-t1)})
where H=H₁
The temperature versus time curve which results from the application of algorithm 110′ in FIG. 7 to a standard liver tissue sample may be expressed by the following equation from time t₂ to t₃:
Tₛ=T₂e^{-(K/Q)(t-t2)}+((H₁+KTₕ)/K)(1−e^{-(K/Q)(t-t2)})+(((H₂−H₁)/(t₃−t₂))/K)(t−t₂)−((H₂−H₁)/(t₃−t₂))Q/K²)(1−e^{-(K/Q)(t-t2)})
where H=H₁+(H₂−H₁)(t−t₂)/(t₃−t₂)
The temperature versus time curve which results from the application of algorithm 110′ in FIG. 7 to a standard liver tissue sample may be expressed by the following equation from time t₃ to t₄:
Tₛ=T₃e^{-(K/Q)(t-t3)}+((H₂+KTₕ)/K)(1−e^{-(K/Q)(t-t3)})
where H=H₂

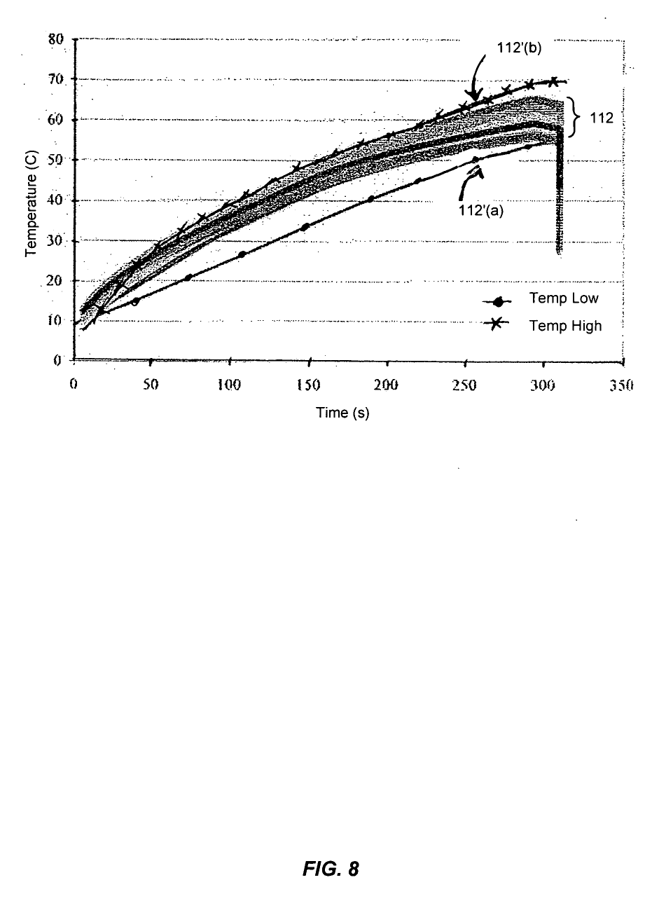

The above equations are graphed for power levels typically used on the liver samples. The theoretically predicted form of the temperature response curve 112′ (depicted by the letter x) is superimposed on the driving RF open loop power algorithm 110′ (depicted by asterisks *) in FIG. 7. Numerous repeated laboratory tests have confirmed a good match between the mathematically predicted temperature curve 112′ and actual observation 112 (FIG. 8). The only variation is in peak temperature, which is observed to vary over a range as would be expected based in the previously discussed variations in the two controlling parameters, K and Q. FIG. 8 illustrates twenty five observed tissue temperature curves 112 achieved from in vitro studies on bovine liver tissues with the open loop power application 110′. Such open loop power methods 110′ result in heating the structural support tissue to the desired temperature range between 55° C. and 65° C., with a mean endpoint tissue treatment temperature of 59° C. The upper theoretically predicted temperature curve 112′(b) is shown, reaching 70° C. for a Q value of 55 joules/° C. and a K value of 0.25 watts/° C. The lower theoretically predicted temperature curve 112′(a) is also shown, reaching 55° C. for a Q value of 85 joules/° C. and a K value of 0.27 watts/° C. The validity of the theoretical mathematical model is thus confirmed.

It will be appreciated that the values of H₁ and H₂ in FIG. 7 should be reduced in liver tissues to obtain the same temperatures as in human subjects. This occurs because the human body has an avenue for heat losses from the treatment zone that is not present in the laboratory liver sample. Additionally, the Q value for human subjects varies more widely than that of the laboratory liver sample. Hence, as already noted above, the temperature versus time equations are finally adjusted to reflect these effects before they can be used to predict actual temperature response range, to any given power algorithm, in human subjects. The following section deals with these relevant human anatomy adjustments.

Human Anatomy Factors

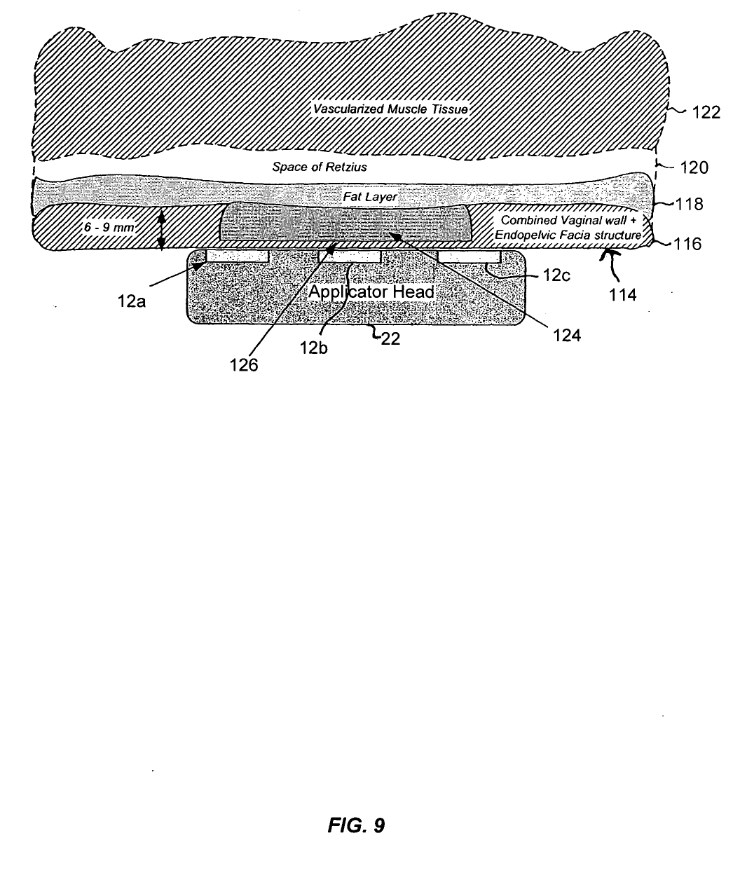

FIG. 9 illustrates a transvaginal noninvasive applicator 22 with electrodes 12a, 12b, 12c positioned against vaginal mucosa intermediate tissue 114 to heat a deeper collegenated tissue in the endopelvic fascia 116. The combined vaginal wall and endopelvic fascia 116 is shown overlying the vaginal mucosa 114. A fat tissue structure 118 providing a thin insulating layer is shown overlying the endopelvic support tissue 116. A variable space, known as the space of retzius 120, is provided between the fat tissue layer 118 and vascularized muscle tissue 122.

FIG. 9 may be compared with FIG. 6 which shows the liver sample being treated in the laboratory as an isolated free body, thermally isolated from its surroundings so that only the applicator head 22 acts upon it. As such, losses of heat due to the environment from the liver during treatment can reasonably be considered negligible. In the human body, however, the tissue being treated is backed by other tissue. Specifically, the vaginal wall and endopelvic fascia structure 116 which is being treated has a layer of fat 118, which along with the space of retzius 120 forms a thermal barrier. This combined with the high thermal mass of the vascularized muscle tissue 122 underneath stops the tissue effects boundary at the back of the endopelvic fascia 116. Although temperatures do not rise significantly beyond this point, there is loss of heat across this boundary, which is simply absorbed by the underlying muscle tissue 122 as if it were an infinite heat sink.

Since the fat layer 118 varies in thickness and the intimacy of contact between the tissue structures across the space of retzius 120 is also variable, the losses of heat from the treatment zone 116 to the underlying tissues 122 is variable from patient to patient and even between sides of a particular patient. Considering the directions radially outward within the endopelvic fascia 116, the thermal gradients form a consistent pattern which is taken into account in the conception of the thermal capacity Q of the volume being treated. Therefore losses in those directions need not be considered. It is the variable losses in the increasing depth dimension that affect outcomes that should be considered. It will further be appreciated that blood circulation within the endopelvic fascia 116 is very minor, so heat transfer can be treated as conduction.

Transfer of heat from the tissue effects volume 124 to the rest of the body is given by the following equation:
L_{B}=D(Ts−37)
L_{B} represents the rate of loss of thermal energy from the treatment zone to the rest of the body. D represents a coefficient of thermal conductivity between the measured point (Ts) in the tissue effects volume 124 and the rest of the body (watts/° C.). The body temperature is naturally regulated at 37° C. The application of this equation is complicated by the fact that the applicator 22 may cool the treatment zone 116 below body temperature before beginning RF treatment to heat it up. Therefore, until Ts moves up and past body temperature, losses are negative as the body is in effect helping the RF in heating the treatment zone. After Ts passes above body temperature, do then losses become positive. At the instant the treatment temperature passes through body temperature, losses are momentarily zero.

Taking into account the rate of loss of thermal energy from the treatment zone to the body (L_{B}), heating to equilibrium at constant RF power in vivo may now be expressed by the following equations:
L_{TOTAL}=L_{A}+L_{B}=K(Tₛ−Tₕ)+D(Tₛ−37)
at equilibrium H=L_{TOTAL}
H=L_{TOTAL}=K(Tₛ−Tₕ)+D(Tₛ−37) or Tₛₑ=(H+KTₕ+37D)/(K+D) (Equation #4)
As discussed above, these equilibrium calculations are insufficient to describe power applications for incontinence treatment because the treatment procedure times for the equilibrium temperature to be reached are lengthy and unpractical. Therefore non-equilibrium effects in vivo need to be considered.

Taking into account the rate of loss of thermal energy from the treatment zone to the body (L_{B}), derivation of temperature versus time for constant RF power application in vivo may now be expressed by the following equations:
Hₙₑₜ=H−L_{A}−L_{B} or Hₙₑₜ=H−K(Tₛ−Tₕ)−D(Tₛ−37)
The instantaneous rate of temperature rise is given by the following equations:
dTₛ/dt=Hₙₑₜ/Q or dT/ds=(H−(K+D)Tₛ+KTₕ+37D)/Q
Solution of this differential equation and application of the boundary condition to temperature=Tₒ yields:
Tₛ=T₀e^{-((K+D)/Q)t}+((H+KTₕ+37D)/(K+D))(1−e^{-((K+D)/Q)t}) (Equation #5)

Taking into account the rate of loss of thermal energy from the treatment zone to the body (L_{B}), derivation of temperature versus time for ramping RF power application in vivo may now be expressed by the following equation:
H=Hₒ+Pt
The instantaneous rate of temperature rise is given by the following equations:
dTₛ/dt=Hₙₑₜ/Q or dTₛ/dt=(H₀+Pt−(K+D)Tₛ+KTₕ+37D)/Q
Solution of this differential equation and application of the boundary condition to temperature=Tₒ yields:
Tₛ=T₀e^{-((K+D)/Q)t}+((H₀+KTₕ+37D)/(K+D))(1−e^{-((K+D)/Q)t})+(P/(K+D))t−(PQ/(K+D)²)(1−e^{-((K+D)/Q)t}) (Equation #6)

Taking into account the rate of loss of thermal energy from the treatment zone to the body (L_{B}), derivation of temperature versus time for a segmented RF power application in vivo may now be expressed by the following heat transfer equation from time t₀ to t₁ (FIG. 7):
Tₛ=T₀e^{-((K+D)/Q)(t-t0)}+((H₀+KTₕ+37D)/(K+D))(1−e^{-((K+D)/Q)(t-t0)})+(P/(K+D))(t−t₀)−(PQ/(K+D)²)(1−e^{-((K+D)/Q)(t-t0)})
where H=H₀+(H₁−H₀)(t−t₀)/(t₁−t₀)
The temperature versus time for a segmented RF power application in vivo may now be expressed by the following heat transfer equation from time t₁ to t₂ (FIG. 7):
Tₛ=T₁e^{-((K+D)/Q)(t-t1)}+((H+KTₕ+37D)/(K+D))(1−e^{-((K+D)/Q)(t-t1)})
where H=H₁
The temperature versus time for a segmented RF power application in vivo may now be expressed by the following heat transfer equation from time t₂ to t₃ (FIG. 7):
Tₛ=T₂e^{-((K+D)/Q)(t-t2)}+((H₁+KTₕ+37D)/(K+D))(1−e^{-((K+D)/Q)(t-t2)})+(P/(K+D))(t−t₂)−(PQ/(K+D)²)(1−e^{-((K+D)/Q)(t-t2)})
where H=H₁+(H₂−H₁)(t−t₂)/(t₃−t₂)
The temperature versus time curve for a segmented RF power application in vivo may now be expressed by the following heat transfer equation from time t₃ to t₄ (FIG. 7):
Tₛ=T₃e^{-((K+D)/Q)(t-t3)}+((H+KTₕ+37D)/(K+D))(1−e^{-((K+D)/Q)(t-t3)})
where H=H₂

Although the effective thermal capacity Q and coefficient D for the temperature versus time equations for an individual treatment can not be predicted, actual response curves from multiple treatments can be used to develop a profile of the range of these figures. The coefficient K is the least variable, and remains very close to 0.26+/−0.01 watts/° C., as previously calculated. K is a function of the interface between the brass applicator head and the tissue, and varies little from treatment to treatment. Q and D however are highly dependent on anatomical variations among patients and even between sides on the same patient.

Thermal capacity Q would be expected to vary with the thickness of the endopelvic fascia 116, which typically varies from 6 to 9 mm as shown in FIG. 9. Thermal capacity Q would also be expected to vary with moisture content and tissue density. Thermal capacity Q is not predicted for an individual treatment, but instead its value is derived for each actual treatment by analysis of the observed response. The value of Q for any particular in vivo treatment can be surmised by direct observation of the temperature time curve for that treatment. At the unique moment when the temperature rises past body temperature (37° C.), losses to the body are instantaneously zero. This means that for this instant, the slope of the temperature time line is given by the following equation:
dTₛ/dt=(H−K(Tₛ−Tₕ))/Q or Q=(H−37K+KTₕ)/(dTₛ/dt) (Equation #7)
H and Tₕ are controlled variables. By observing the slope of the temperature line and the instantaneous power being applied as the temperature rises through body temperature, the required information to calculate effective Q for that particular treatment may be obtained.

Thermal transfer coefficient between the treatment zone and the rest of the body D would be expected to vary with the intimacy of contact between the endopelvic fascia 116 and the underlying muscular tissue 122. This varies with the thickness of the fat layer 118 behind the endopelvic fascia 116 and the size of the space of retzius 120. The peak temperature equations described above may be plotted using a plotting program, such that the theoretical curves resulting from the various values of the three coefficients, K, Q, and D may be readily observed. With the values of K set at nominal, and the value of Q determined from an actual observed temperature plot, iterative curve fitting may be utilized to estimate the value of D which would produce the observed in vivo temperature plot. By this means, the values for Q and D may be determined for the open loop power treatments of the present invention, for which temperature was monitored. The derived Q value was in a range from 40 joules/° C. to 87 joules/° C. and the derived D value was in a range from 0.39 watts/° C. to 1.19 watts/° C.

These extreme values have been applied to the heat transfer equations to calculate projected highest and lowest peak temperatures which will be seen with the open loop algorithm of the present invention. This process adds a very large measure of conservatism, because it is assumed that the three most extreme values coincide in the same treatment. Since the coinciding of these extreme values in a single treatment is itself an extremely unlikely occurrence, the calculated values for the temperature extremes are very rare. The inherent mathematics of the heat transfer equations leads to a non-normal distribution of peak temperatures. The distribution is skewed towards the lower temperatures. Therefore, although the average values for the coefficients are known, average peak temperature may not be reliably calculated. Fortunately, only the range is needed to select an algorithm. Since it has been observed with appropriate algorithm characteristics, such as gradual power ramp up and sufficient dwell time, efficacy performance is very forgiving of peak temperature variation. Hence, the objective of this calculation becomes mainly the assurance that the expected range of peak temperatures falls within the allowable range for safety, while allowing the algorithm to run long enough, and while favoring the high end of the temperature range for efficacy. In sum, a tight temperature range is not necessary.

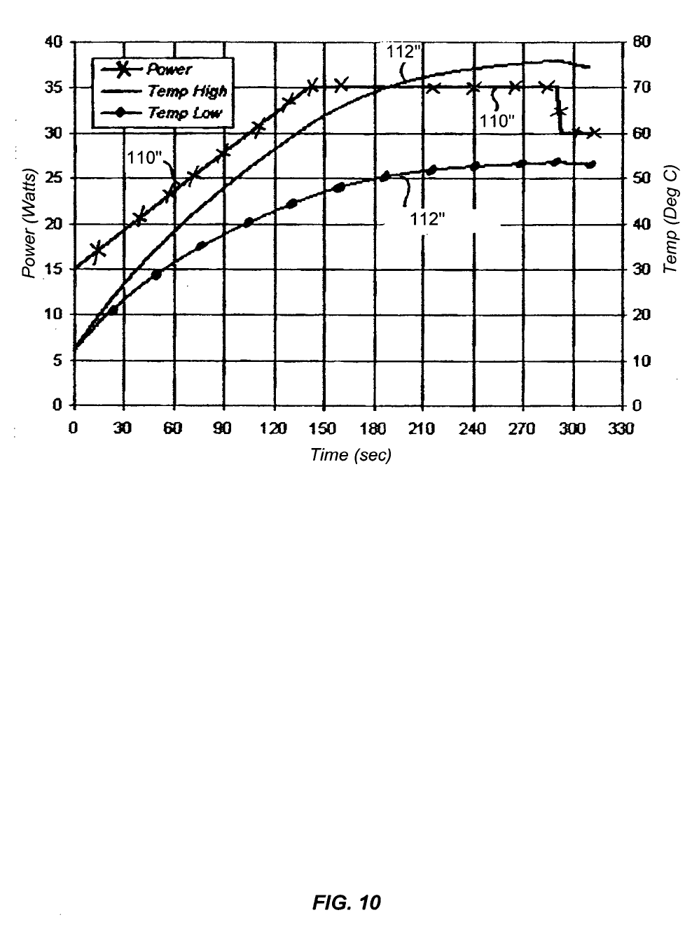

FIG. 10 illustrates the theoretical projection range of peak temperatures 112″ for a chosen open loop power algorithm 110″ applied to a patient population. The chosen open loop RF power algorithm 110″ is described as follows: (1) pre-cool down period brings treatment zone starting temperature to approximately 12° C.; (2) RF power starts at 15 watts and ramps at 0.143 watts/second for 140 seconds to reach 35 watts (step 102); (3) RF power then dwells at 35 watts for 150 seconds (step 104); (4) RF power then drops rapidly to 30 watts over 3 seconds (step 106); (5) RF power then holds at 30 watts for 17 seconds (step 108); (6) RF power then shuts off and applicator is held in place for a 30 second post-cooling period. So, for this power algorithm 110″ in the heat transfer equations, the variables H1=35; H2=30; T₀=12° C.; Tₕ=0° C.; t₀=0; t₁=140; t₂=290; t₃=293; and t₄=310. The conservative low and high values derived above for the coefficients K, Q, and D were also put into the heat transfer equations.

As shown in FIG. 10, the theoretically predicted highest and lowest peak temperatures 112″ are 76° C. and 54° C., respectively, which would be expected to be observed when this open loop power algorithm 110″ is applied to the target population. As mentioned earlier, the average value can not be accurately calculated, but the mid-point between the high and low values is 65° C. Since treatment has been shown to be safe at any temperature below 78° C., and since it appears to be quite effective over almost the entire range from 50° C. to 78° C., the choice of algorithm is appropriate.

Experimental

The following description of experimental studies provides some specific, non-limiting examples that are encompassed by the present invention.

A series of experiments using a Sol2 head to treat 10 mm thick pieces of bovine liver were performed using the open loop power algorithms 110 of the present invention. At the conclusion of each treatment the safety zone thickness 126 (FIG. 9), as well as the length, width and depth of the treatment zone (tissue treatment volume 124, FIG. 9) were measured based on the color change in the liver, which occurs at about 50° C. This volume approximates the volume of tissue where necrosis would occur in living tissue. Following each series of 25 treatments, the composite temperature distribution was plotted and compared to theoretical predictions from the mathematical model described above.

Experiments were run using two comparison treatment algorithms. A Sol1 power step algorithm was run with its initial 24 second treatment at 20 watts followed by treatment at 41 watts. This value was chosen to achieve a mean treatment time on liver close to that seen in a Sol1 clinical study (109 seconds). The Sol1 power step algorithm treated until tissue temperature reached 70° C. or 150 seconds of treatment had occurred.

The Sol2 open loop power algorithm 110 was used with power levels chosen to achieve mean treatment temperatures of 59° C., 68° C., or 75° C. Each series involved 25 treatments. The Sol2 algorithm starts at 15 watts and rises to full treatment power after 140 seconds. It remains at that power level for 150 seconds and then drops by 5 watts for a 20 second dwell period. The 59° C. mean treatment temperature matches the mean temperature observed in open loop Mexico feasibility patients. These patients received full treatment power for only 60 seconds. The higher liver treatment target of 68° C. was chosen to be slightly larger in order to provide a worst case estimate of the expected safety zones in human patients. The 75° C. treatments were done to provide a worst case lower limit to the safety zone thickness.

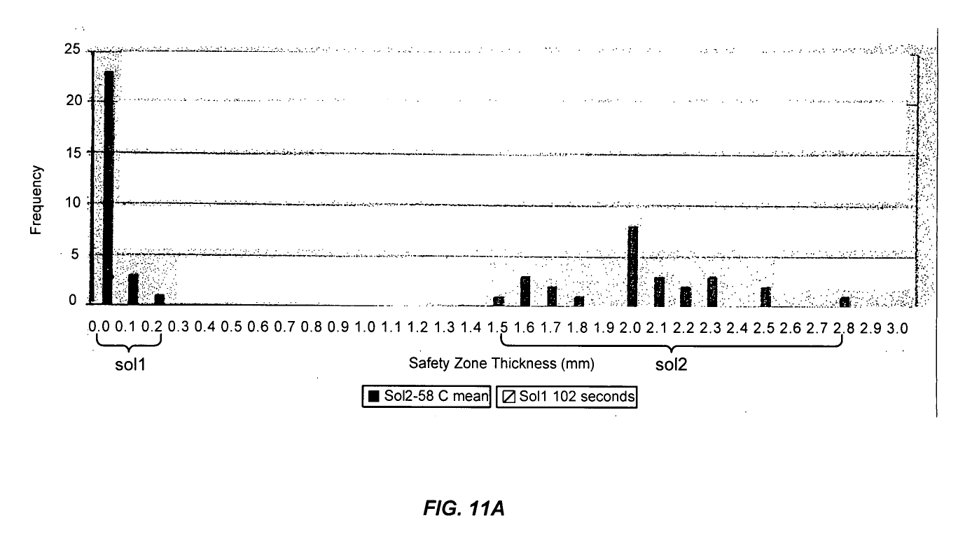

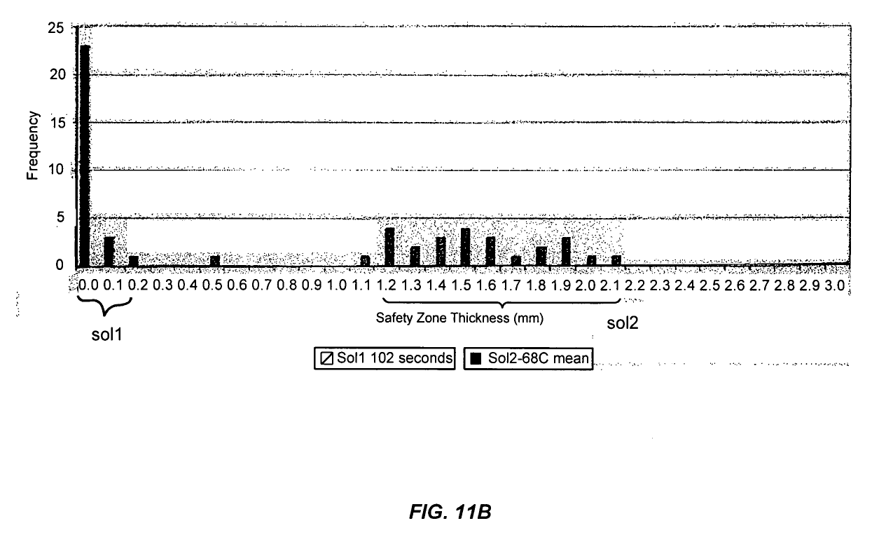

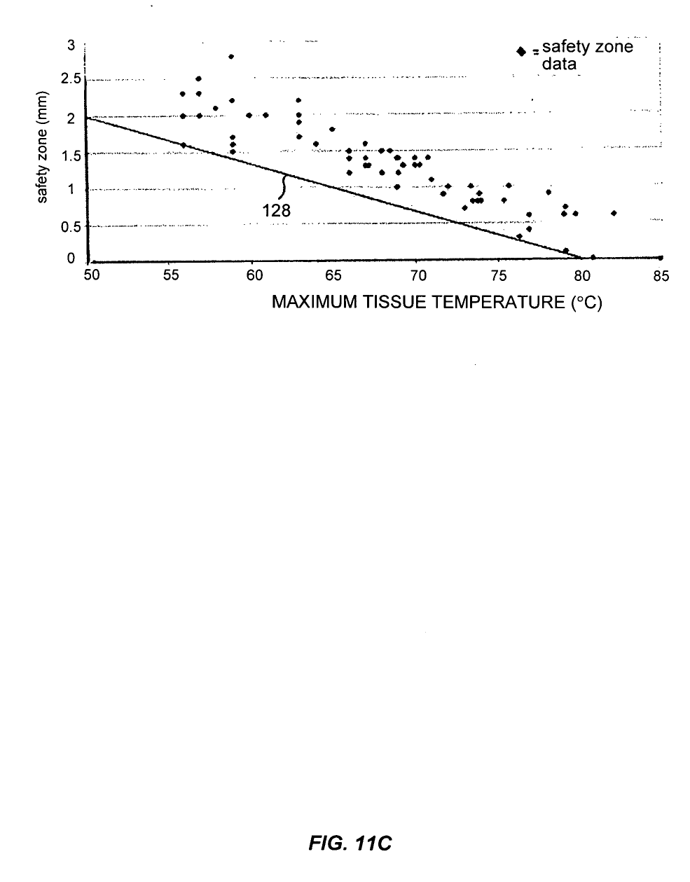

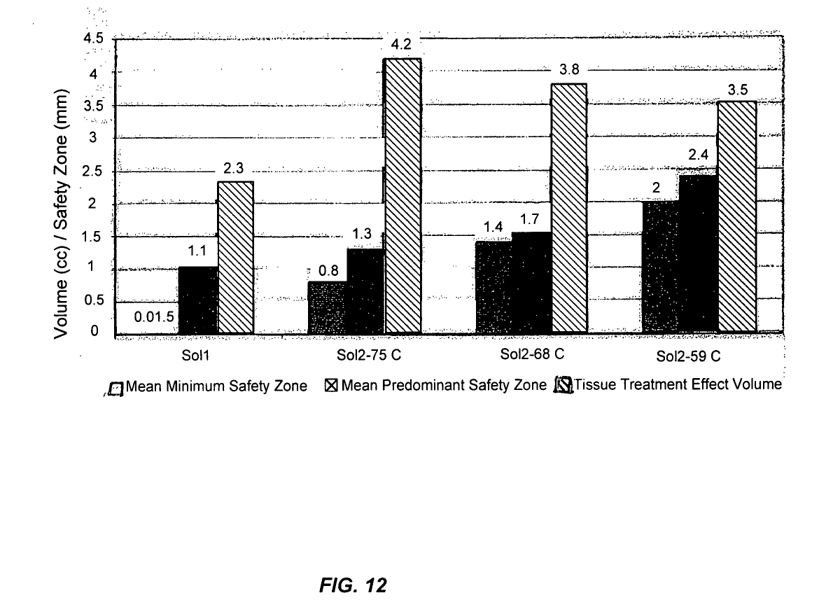

As illustrated in FIGS. 11A through 11C, the Sol2 open loop power algorithm 110 increases the mean minimum safety zone thickness to 2.0 mm at the 59° C. treatment (FIG. 11A) and to 1.4 mm at the 68° C. treatment (FIG. 11B) versus the 0.01 mm mean minimum value measured using the Sol1 power step algorithm. As shown in FIGS. 11C and 12, even when the treatment temperature was increased to 75° C., the mean minimum safety zone was still 0.8 mm. The safety zone thickness in Sol2 represents a substantial increase over that produced by the Sol1 power algorithm. FIG. 11C plots the safety zone data from 75 treatments against tissue temperature, where line 128 denotes the safety zone lower limit. These temperature ranges represent the maximum predicted value for any patient in the proposed Sol2 clinical study and indicates that the vaginal mucosal surface will be preserved. The safety zone and tissue effects volume values are further compared in FIG. 12. The predominant safety zone thickness ranges from 1.3 mm at the 75° C. treatment to 2.4 mm at the 59° C. treatment in the Sol2 case still exceeds the 1.1 mm Sol1 value. Significantly, both the minimum safety zone and the predominant safety zone values easily meet the requirements of 0.5 mm and 1.0 mm respectively known to allow in vivo use without adverse thermal effects.

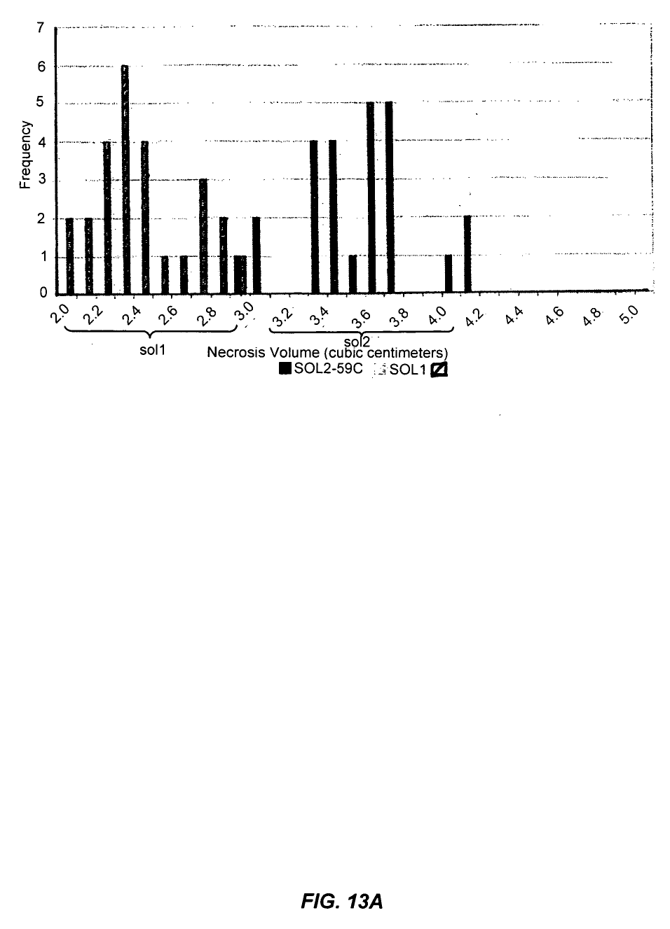

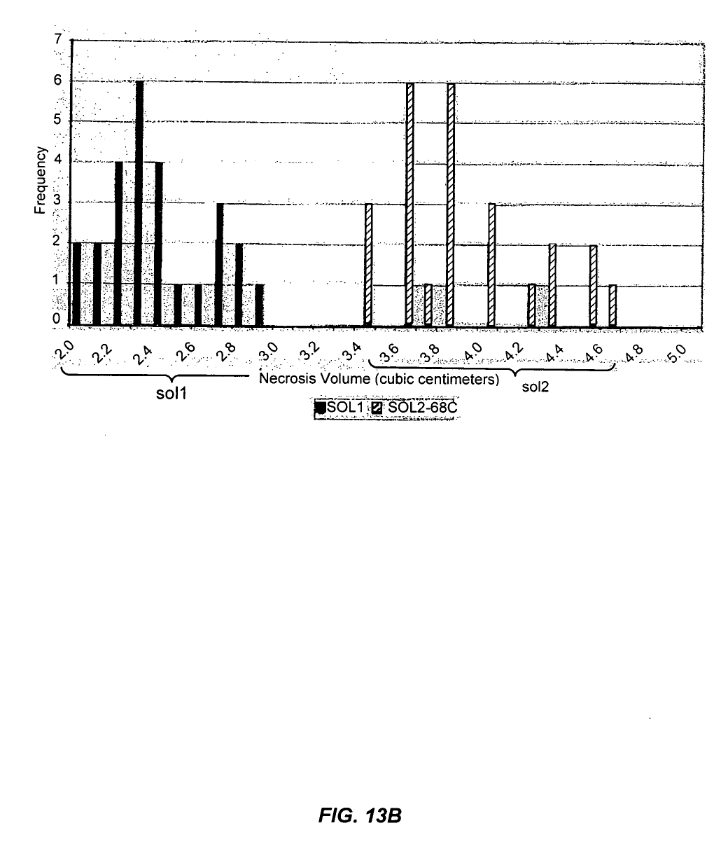

FIGS. 13A and 13B illustrate further tissue treatment volume results from the experimental temperature studies. The necrosis volume is only 2.3 cubic centimeters for Sol1 but increases by 52% to 3.5 cubic centimeters for Sol2 at 59° C. treatment and by 65% to 3.8 cubic centimeters for Sol2 at 68° C. treatment and by 83% to 4.2 cubic centimeters for Sol2 at 75° C. treatment. This increase in treated tissue volume is expected to increase the efficacy of the procedure since a larger volume of tissue is strengthened and stiffened following the healing process and is therefore expected to provide improved urethral support. It is believed that the ramp shape of the open loop power algorithm of Sol2 provides the substantially larger treatment volume versus the step function in Sol1. This increased treatment volume raises the efficacy. It will be appreciated that the above measured volumes have been calculated based on a rectangular shaped treatment zone. The actual treatment zone for incontinence treatment comprises a cylindrical or conical shaped treatment zone, and as such the actual treatment volumes may be 35% to 50% of those measured using the rectangular shaped treatment zone approximation. As such, actual tissue treatment volumes may be in a range from 1 cubic centimeters to 5 cubic centimeters.

A separate series of experiments were run to measure the K value and the Q value for use in comparing the theoretical curves to the observed tissue temperature curves in liver. The liver samples in all experiments were covered with saran wrap on the untreated side. A melamine thermal insulation box was placed over the tissue to minimize air losses. As discussed in detail above, the following equation:
Tₛₑ=(H/K)+Tₕ
is used to calculate K. The equilibrium tissue temperature is measured by performing 30 to 50 minute treatments at low power values. FIG. 14 shows the equilibrium experiment based on an average of 6 treatments. The mean K value of 0.26 watts/° C. was determined with a standard deviation of less than 0.01 watts/° C.

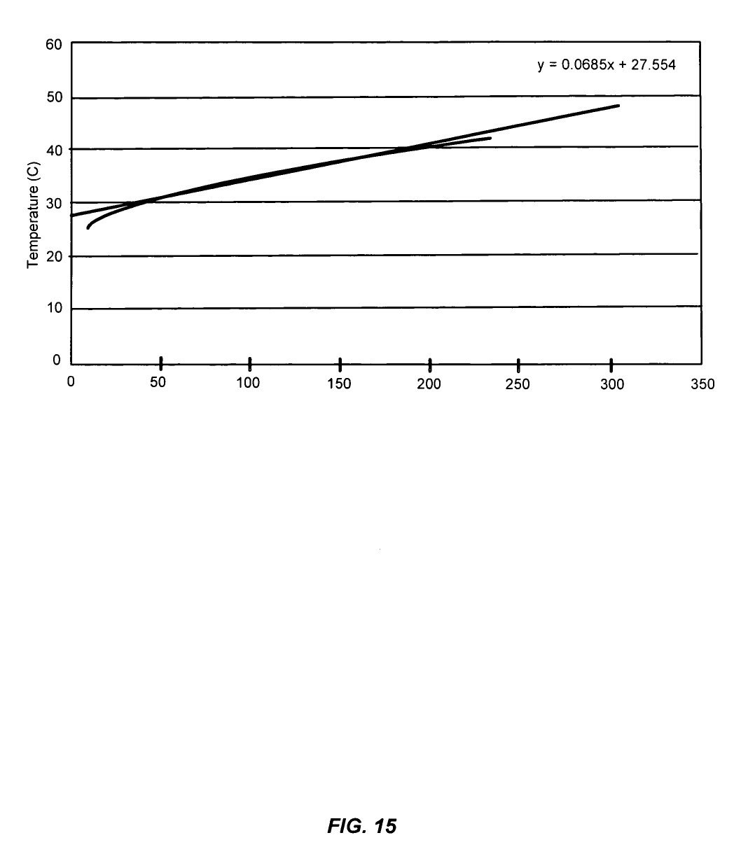

As discussed in detail above, the following equation is used to calculate Q:
dTₛ/dt=H/Q or Q=H/slope
The tissue temperature slope is measured using short treatment times with a non-cooled applicator head. The heating power is in the range from 5 to 7 watts which matches the net RF heating rate (35 watt heating less 28 to 30 watts of cooling) during an actual treatment. The Q value was measured by averaging the temperature rise curve in six different runs. FIG. 15 shows a straight line fit to the curve indicating a Q value of 70±15 joules/° C.

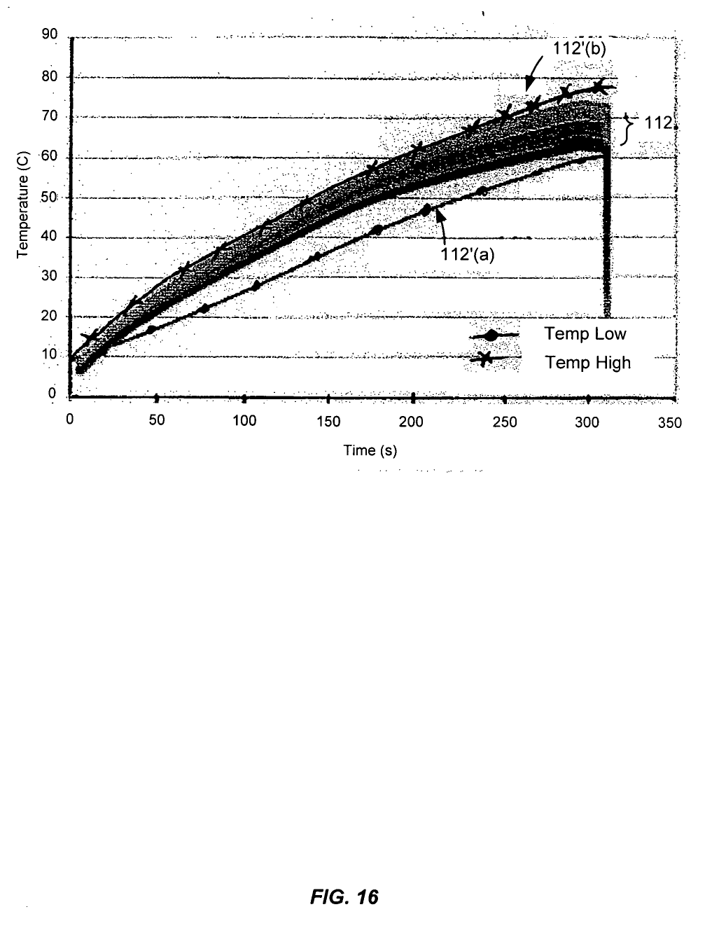

The composite curves for the 25 treatment runs are shown in FIGS. 8 and 16. In FIG. 8, the observed mean treatment temperature value was 59° C. with a standard deviation of 2.5° C. In FIG. 16, the twenty five observed tissue temperature curves 112 achieved from open loop power methods 110′ result in heating the structural support tissue to the desired temperature range between 63° C. and 73° C., with a mean endpoint tissue treatment temperature of 68° C. and a standard deviation of 3.1° C. The upper theoretically predicted temperature curve 112′(b) is shown, reaching 77° C. for a Q value of 55 joules/° C. and a K value of 0.25 watts/° C. The lower theoretically predicted temperature curve 112′(a) is also shown, reaching 61° C. for a Q value of 85 joules/° C. and a K value of 0.27 watts/° C. The validity of the theoretical mathematical model is thus confirmed.

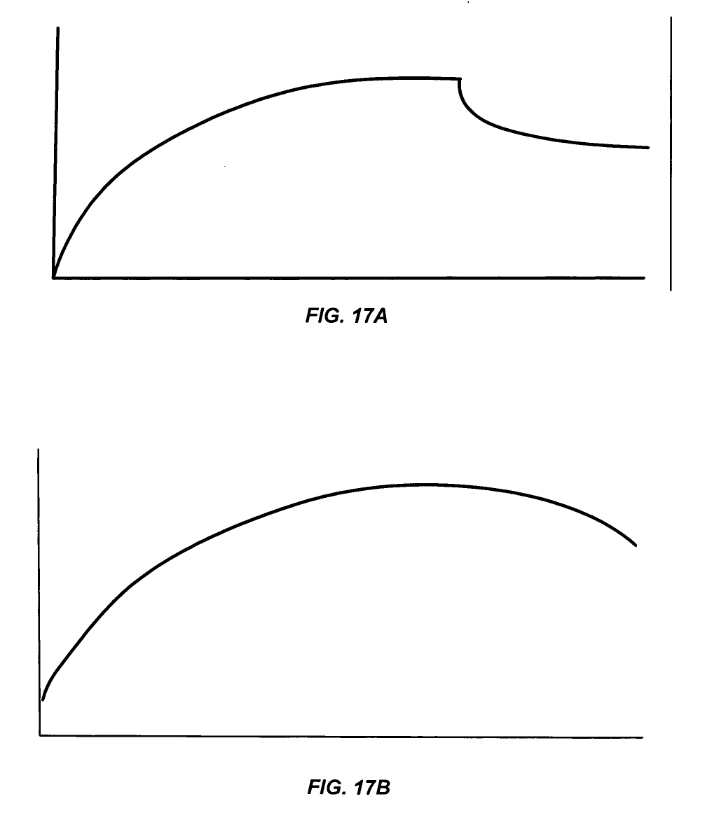

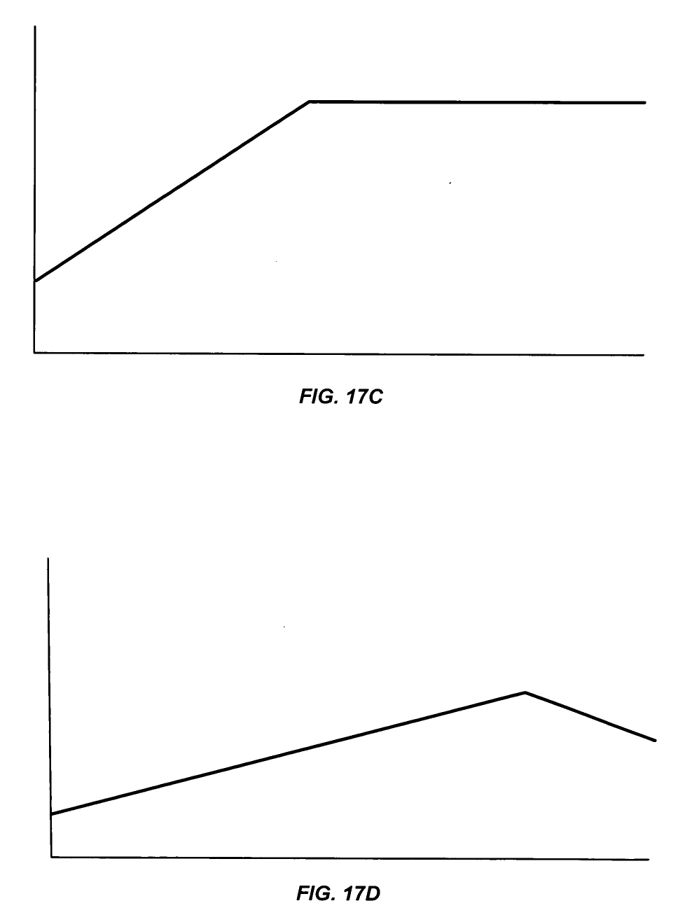

FIGS. 17A through 17D illustrate alternative power profiles that may be utilized to achieve results similar to those of the present invention. In particular, the concepts of the present invention may be applied to create variations on the algorithm form using curves instead of straight lines to achieve similar results. FIGS. 17A through 17D are some examples of sample shapes that may be utilized to achieve similar results to the preferred algorithm shape (FIG. 10). FIG. 17A shows a two hyperbola form, FIG. 17B shows a truncated parabola shape, FIGS. 17C and 17D illustrate partial straight line algorithm variants.

Although certain exemplary embodiments and methods have been described in some detail, for clarity of understanding and by way of example, it will be apparent from the foregoing disclosure to those skilled in the art that variations, modifications, changes, and adaptations of such embodiments and methods may be made without departing from the true spirit and scope of the invention. Therefore, the above description should not be taken as limiting the scope of the invention which is defined by the appended claims.

What is claimed is:

1. A method for therapeutically heating a collagenous structural support tissue of a pelvic support system to a desired temperature range, the method comprising:
delivering energy to the structural support tissue to heat the tissue to the desired temperature range by:
ramping up a power level for a first period of time;
maintaining a first constant power level for a second period of time;
ramping down the power level for a third period of time; and
maintaining a second constant power level for a fourth period of time.

2. The method of claim 1, wherein ramping up the power level for the first period of time comprises ramping up an initial power level that is less than about 22 watts at a slope that is less than about 0.5 watts per second.

3. The method of claim 2, wherein the first period of time is in a range from 50 seconds to 220 seconds.

4. The method of claim 2, wherein the first constant power level is higher than the second constant power level.

5. The method of claim 2, wherein first constant power is in a range from 34 watts to 40 watts and the second period of time is in a range from 60 seconds to 200 seconds.

6. The method of claim 5, wherein the ramping down the power level for the third period of time comprises ramping down the power level to a range from 29 watts to 33 watts at a slope in a range from 0.5 watts per second to 20 watts per second.

7. The method of claim 6, wherein the third period of time is less than about 3 seconds.

8. The method of claim 6, wherein the second constant power is in a range from 29 watts to 33 watts and the fourth period of time is in a range from 15 seconds to 120 seconds.

9. The method of claim 1, wherein the structural support tissue is heated to the desired temperature range between 54° C. and 76° C.

10. The method of claim 1, wherein energy delivery produces a mean minimum safety zone thickness in an intermediate tissue of at least 0.3 mm.

11. The method of claim 1, wherein energy delivery produces a mean predominant safety zone thickness in an intermediate tissue of at least 0.5 mm.

12. The method of claim 1, wherein energy delivery produces a tissue treatment volume in a range from 1 cubic centimeters to 5 cubic centimeters.

13. The method of claim 12, wherein an effective thermal capacity of the tissue treatment volume is in a range from 40 joules/° C. to 87 joules/° C.

14. The method of claim 12, wherein a coefficient of thermal conductivity between a measured point in the tissue treatment volume and a non-treated tissue is in a range from 0.39 watts/° C. to 1.19 watts/° C.

15. The method of claim 12, wherein a coefficient of thermal conductivity between a measured point in the tissue treatment volume and an applicator body is in a range from 0.2 watts/° C. to 0.35 watts/° C.

16. The method of claim 1, further comprising pre-cooling the structural support tissue.

17. The method of claim 1, wherein the energy is delivered so as to effect shrinkage of the structural support tissue.

18. The method of claim 1, wherein the energy is delivered so as to cause bulking and buttressing of the structural support tissue during healing.

19. The method of claim 17 or 18, wherein the shrinkage or tissue bulking/buttressing inhibit urinary incontinence or bladder neck descent.

20. The method of claim 1, wherein the structural support tissue comprises a collegenated tissue in an endopelvic fascia.

21. The method of claim 1, further comprising accessing the structural support tissue transvaginally.

22. The method of claim 1, further comprising accessing the structural support issue laparoscopically.

23. The method of claim 1, wherein the energy comprises radio frequency energy.

24. The method of claim 1, wherein the delivering is automatically carried out by a processor.

25. A system for therapeutically heating a collagenous structural support tissue of a pelvic support system to a desired temperature range, the system comprising:
an applicator body;
a processor coupleable to the applicator body, the processor programmed to deliver energy to the structural support tissue with the applicator body by ramping up a power level for a first period of time, maintaining a first constant power level for a second period of time, ramping down the power level for a third period of time, and maintaining a second constant power level for a fourth period of time

26. The system of claim 25, further comprising a power supply coupleable to the processor.

27. The system of claim 25, further comprising a cooling source coupleable to the processor.

28. A computer-readable storage medium having a computer-readable program embodied therein for directing operation of a computer system, the computer system including a communications system, a processor, and a memory device, wherein the computer-readable program includes instructions for therapeutically heating a collagenous structural support tissue of a pelvic support system to a desired temperature range in accordance with the following:
delivering energy to the structural support tissue to heat the tissue to the desired temperature range by:
ramping up a power level for a first period of time;
maintaining a first constant power level for a second period of time;
ramping down the power level for a third period of time; and
maintaining a second constant power level for a fourth period of time.
